(19) United States
(12) Patent Application Publication
(10) Pub. No.: US 2024/0130940 A1
(43) Pub. Date: Apr. 25, 2024

(54) AN IMPROVED LONG WEARING SUNSCREEN COSMETIC

(71) Applicant: BASF SE, Ludwigshafen am Rhein (DE)

(72) Inventors: Yan LIU, Shanghai (CN); Juan CHU, Shanghai (CN); Xiao Ying HU, Shanghai (CN)

(21) Appl. No.: 18/277,596

(22) PCT Filed: Feb. 10, 2022
(86) PCT No.: PCT/EP2022/053267

§ 371 Date: Aug. 16, 2023

(30) Foreign Application Priority Data
Feb. 18, 2021 (WO) PCT/CN2021/076732

(51) Int. Cl. A61K 8/37; A61K 8/25; A61K 8/368; A61K 8/41; A61K 8/49; A61K 8/81; A61Q 17/04

(52) U.S. Cl. CPC A61K 8/37; A61K 8/25; A61K 8/368; A61K 8/411; A61K 8/4966; A61K 8/8152; A61Q 17/04; A61K 2800/30

(57) ABSTRACT

The present invention relates to a sunscreen composition, which comprises 5 to 50% by weight of at least one UV protective agent; 0.05 to 5% by weight of at least one hydrophobically modified clay mineral; 1 to 60% by weight of at least one oil component; and 0.1 to 4% by weight of at least one associative polymer. The present invention also provide a method of achieving an improved sunscreen protection effect with desirable film morphology and excellent wear properties.

### TECHNICAL FIELD

The present invention relates to the field of sunscreen composition, i.e. composition which, when applied to the skin, provide protection against the damaging effects of ultraviolet radiation. More particular, the invention provides a sunscreen composition which provides long wearing and water-resistant film. More particular, the invention relates to a method of achieving improved long wearing and water-resistant sunscreen effect by applying the composition onto the skin.

### BACKGROUND ART

In recent years, the general public has become more and more aware of the damaging effects of ultraviolet radiation from the sun. Consequently, sunscreen composition has become popular for people who spend time in the sun.

A challenge in formulating a sunscreen composition is to produce a composition with good substantivity, i.e. retention on the skin. Certainly, a sunscreen composition effectiveness is tied to its ability to stay in place on the skin of the user for the entire time the user is in the sun. As a result, it is important that the composition can be resistant to being rubbed off or washed off in water.

In recent times there is still an increasing demand for sunscreen composition which can form a fully integrated film, leading to improved wear properties such as long-lasting, good resistance to water and abrasion, and anti-dust effect.

WO 2016/068298 A1 discloses a water-in-oil emulsion sunscreen cosmetic which provides an improved UV protection due to contact with water and/or sweat compared to directly after application and which is also pleasant to use and easy to wash off. However, the disclosed sunscreen composition cannot achieve desirable resistance properties to water and abrasion.

In view of the aforementioned problems, it is thus the object of the present invention to provide a sunscreen composition which have advantages of ultraviolet protection effect with desirable wear properties, such as long-lasting and good resistance to water, abrasion and dust.

### SUMMARY OF INVENTION

In one aspect, the present invention is directed to a sunscreen composition comprising:
a) from 5 to 50% by weight of at least one UV protective agent;
b) from 0.05 to 5% by weight of at least one hydrophobically modified clay mineral;
c) from 1 to 60% by weight of at least one oil component;
d) from 0.1 to 4% by weight of at least one associative polymer.

In one aspect, the sunscreen compositions of the present invention comprise an associative polymer which is selected from the group consisting of silicone acrylate polymer; a copolymer of (meth)acrylic acid ester of ethoxylated fatty alcohol and one or more monomers of acrylic acid, methacrylic acid or one of their simple ester; a copolymer of itaconic acid ester of ethoxylated fatty alcohol and one or more monomers of acrylic acid, methacrylic acid or one of their simple ester; and a copolymer based on acrylamidomethylpropanesulphonic acid and (meth)acrylic ester of ethoxylated fatty alcohol. Preferably, the associative polymer is selected from the group consisting of a copolymer of (meth)acrylic acid ester of ethoxylated fatty alcohol and one or more monomer s of acrylic acid, methacrylic acid or one of their simple ester; a copolymer of itaconic acid ester of ethoxylated fatty alcohol and one or more monomers of acrylic acid, methacrylic acid or one of their simple ester; and a copolymer based on acrylamidomethylpropanesulphonic acid and (meth)acrylic ester of ethoxylated fatty alcohol; wherein the ethoxylated (EO) fatty alcohol in each case comprises 5 to 35, in particular 7 to 30, more in particular 7 to 25 ethylene oxide (EO) radicals; More preferably, the sunscreen composition of the present invention comprises an associative polymer which is selected from the group consisting of acrylates/beheneth-25 methacrylate copolymer, acrylates/steareth-20 methacrylate copolymer, acrylates/steareth-20 methacrylate crosspolymer, acrylated/steareth-20 itaconate copolymer; acrylate/ceteth-20 itaconate copolymer and acrylates/palmeth25 itaconate copolymer, ammonium acryloyldimethyltaurate/beheneth-25-methacrylate copolymer, ammonium acryloyldimethyltaurate/beheneth-25 methacrylate crosspolymer, ammonium acryloyldimethyltaurate/Steareth-25 methacrylate crosspolymer, ammonium acryloyldimethyltaurate/steareth-8 methacrylate copolymer and ammonium acryloyldimethyltaurate/laureth-7 methacrylate copolymer and a combination thereof.

In one aspect, the sunscreen composition of the present invention comprises the UV protective agent which is selected from the group consisting of ethylhexyl methoxycinnamate (Uvinul® MC80), ethylhexyl triazone (Uvinul® T150), methylene bis-benzotriazolyl tetramethylbutylphenol (Tinosorb® M), bis-ethylhexyloxyphenol methoxyphenyl triazine(Tinosorb®S), diethylamino hydroxybenzoyl hexyl benzoate (Uvinul® A Plus Granular), ethylhexyl salicylate (Neo Heliopan OS) and a combination thereof.

In a further aspect, the sunscreen composition of the present invention comprises the hydrophobically modified clay mineral which is selected from the group consisting of stearalkonium bentonite, dimethyldistearammonium hectorite, disteardimonium hectorite, dimethylalkylammonium hectorite, benzyldimethylstearylammonium hectorite, magnesium aluminum silicate treated with distearyldimethylammonium chloride and a combination thereof, preferably disteardimonium hectorite.

In another aspect, the sunscreen of the present invention is an emulsifier-free sunscreen composition.

In still another aspect, the present invention provides a method of achieving an improved sunscreen protection effect, comprising applying the sunscreen composition of the present invention.

In still another aspect, the present invention refers to use of the sunscreen composition of the present invention for sunscreen protection of the skin.

Surprisingly and unexpectedly, the application has now discovered that the sunscreen composition of the present invention provides a desirable UV protection effect by forming a fully integrated film with desirable film morphology, which leads to improved wear properties such as long-lasting, excellent resistance to water, abrasion and dust, and thus, makes it possible to achieve the objective as outlined above.

### DESCRIPTION

Throughout the description, including the claims, the term “comprising one” or “comprising a” should be understood as being synonymous with the term “comprising at least one”, unless otherwise specified, and “between” should be understood as being inclusive of the limits.

The terms “a”, “an” and “the” are used to refer to one or to more than one (i.e., to at least one) of the grammatical object of the article.

The term “and/or” includes the meanings “and”, “or” and also all the other possible combinations of the elements connected to this term.

The term “% by weight” used herein, refer to the percent of the total weight of the composition.

It should be noted that in specifying any range of concentration, weight ratio or amount, any particular upper concentration, weight ratio or amount can be associated with any particular lower concentration, weight ratio or amount, respectively.

As used herein, the term “A sunscreen composition” is meant to a composition for topical application to sun-exposed areas of the skin and/or hair of mammals, especially humans. Such a composition may be generally classified as leave-on or rinse off, and includes any product applied to a human body for also improving appearance, cleansing, odor control or general aesthetics. It is more preferably a leave-on product. The composition of the present invention can be in the form of a liquid, lotion, cream, foam, scrub, gel, or toner, or applied with an implement or via a face mask, pad or patch. Non-limiting examples of such sunscreen compositions include leave-on lotions, creams, antiperspirants, deodorants, lipsticks, foundations, mascara, sunless tanners and sunscreen lotions. “skin” as used herein is meant to include skin on the face and body (e.g. neck, chest, back, arms, underarms, hands, legs, buttocks and scalp) and especially to the sun exposed parts thereof. The composition of the invention is also of relevance to applications on any other keratinous substrates of the human body other than skin e.g. hair where products may be formulated with specific aim of providing photoprotection.

As used herein, the term “associative polymer” means any amphiphilic polymer comprising at least one fatty chain, i.e., a hydrophobic portion and at least one hydrophilic portion.

As used herein, the term “emollient” refers to materials used for prevention or relief of dryness, or for the protection of the skin.

As used herein, the term “emulsifier—free” used herein, is meant to a composition having substantially no emulsifier, which refers to a composition comprises less than about 1 wt % emulsifier agent based on the total weight of the composition.

Unless defined otherwise, all technical and scientific terms used herein have the same meaning as commonly understood by one of ordinary skill in the art to which this invention belongs.

All percentages, parts and ratios are by weight, unless otherwise specified. All such weights as they pertain to listed components are based on the specific ingredient level and, therefore, do not include carriers or by-products that may be included in commercially available materials, unless otherwise specified.

The present invention is directed to a sunscreen composition comprising:
a) from 5 to 50% by weight of at least one UV protective agent;
b) from 0.05 to 5% by weight of at least one hydrophobically modified clay mineral;
c) from 1 to 60% by weight of at least one oil component;
d) from 0.1 to 4% by weight of at least one associative polymer.

UV Protective Agent

The sunscreen composition of the present invention includes at least one or a combined UV protective agent (also known as UV filter), at a concentration, by weight, of greater than 5%, or alternatively about 5% to 50%, or alternatively about 5% to 20%, 25%, 30%, 35%, 40% or 45% based upon weight of the composition. Suitable UV protective agent for the present invention consists of at least one selected from UV absorbing agents and UV scattering agents, which are usually blended in a conventional sunscreen composition. The UV absorbing agents used in the present invention is not specifically limited, and examples thereof include, but not limited to, butyl methoxydibenzoylmethane, ethylhexyl triazone, drometrizole trisiloxane, benzophenone-3, diethylhexyl butamido triazone, bis-ethylhexyloxyphenol methoxyphenyl triazine, 4-methylbenzilidene camphor, and/or benzoxazole compounds, octocrylene, dimethicodiethyl benzalmalonate, polysilicone-15, t butylmethoxydibenzoylmethane, ethylhexyl triazone, hexyl diethylaminohydroxybenzoylbenzoate, bisethylhexyloxyphenol methoxyphenyl triazine, oxybenzone-3, methylene bis-benzotriazolyl tetramethylbutylphenol, phenylbenzimidazolesulfonic acid, homosalate, and ethylhexyl salicylate. The UV scattering agent used in the present invention is not specifically limited, and examples thereof include fine particles of metal oxides such as zinc oxide, titanium oxide, iron oxide, cerium oxide and tungsten oxide. In some embodiments, the UV filters in the present invention is at least one selected from a UV absorbing agent and a UV scattering agent. In one embodiment, the UV filter of the present invention consists only UV absorbing agent. In other embodiment, the UV filter of the present invention consists only UV scattering agent. Each of the abovementioned UV filter amount in the present invention is represented as the total amount of UV absorbing agent and UV scattering agent, and it is particularly preferred to set the amount of UV absorbing agent to 6% or more based upon the total weight of the composition.

As examples, UV Filters (also known as UV protective agent) may be designed below under their INCI name:

Dibenzoylmethane Compounds:
Butylmethoxydibenzoylmethane sold especially under the tradename Parsol 1789 by DSM Nutritional Products, Inc.; Isopropyldibenzoylmethane.

Para-Aminobenzoic Compounds:
Ethyl PABA,
Ethyl Dihydroxypropyl PABA,
Ethylhexyl Dimethyl PABA sold under the name Escalol 507 by ISP,
Glyceryl PABA

Salicylic Derivatives:
Homosalate sold under the commercial name Eusolex HMS by Rona/EM Industries, Ethylhexyl Salicylate sold under the commercial name Neo Heliopan OS by SYMRISE,

Cinnamic Derivatives:
Ethylhexyl Methoxycinnamate sold under the commercial name ARSOL MCX by DSM Nutritional Products,
Isopropyl Methoxy cinnamate,
Isoamyl Methoxy cinnamate sold under the commercial name Neo Heliopan E1000 by SYMRISE, Cinoxate,
Diisopropyl Methylcinnamate,
Derivatives of β,β-diphenylacrylate
Octocrylene sold under the commercial name UVINUL N539 by BASF,
Etocrylene sold under the commercial name UVINUL N35 by BASF,

Benzophenone Derivatives:
Benzophenone-1 sold under the commercial name UVINUL 400 by BASF,
Benzophenone-2 sold under the commercial name UVINUL D50 by BASF,
Benzophenone-3 or Oxybenzone, sold under the commercial name UVINUL M40 by BASF,
Benzophenone-6 sold under the commercial name Helisorb 11 by Norquay,
Benzophenone-8 sold under the commercial name Spectra-Sorb UV-24 by American Cyanamid

Benzophenone-12
n-hexyl 2-(4-N,N-diethylamino-2-hydroxybenzoyl)benzoate sold under the commercial name UVINUL A Plus or in the form of mixture with octylmethoxycinnamate under the commercial name UVINUL A Plus B by BASF,

Benzylidenecamphor Derivatives:
3-Benzylidene camphor manufactured under the commercial name MEXORYL SD by CHIMEX, 4-Methylbenzylidene camphor sold under the commercial name EUSOLEX 6300 by MERCK, Polyacrylamidomethyl Benzylidene Camphor manufactured under the commercial name MEXORYL SW by CHIMEX,

Phenyl Benzotriazole Derivatives:
Drometrizole Trisiloxane sold under the commercial name Silatrizole by RHODIA CHIMIE,
Triazine Derivatives
Bis-Ethylhexyloxyphenol Methoxyphenyl Triazine sold under the commercial name TINOSORB S by BASF,
Ethylhexyl triazone sold under the commercial name UVINUL T150 by BASF,
Diethylhexyl Butamido Triazone sold under the commercial name UVASORB HEB by SIGMA 3V, -triazine silicones substituted by two aminobenzoates groups as those described in the patent EP 0841341, in particular 2,4-bis-(n-butyl 4′-aminobenzalmalonate)-6-[(3-{1,3,3,3-tetramethyl-1-[(trimethylsilyloxy]disiloxanyl})propyl)amino]-s-triazine.

Anthranilic Derivatives:
Menthyl anthranilate sold under the commercial name NEO HELIOPAN MA by SYMRISE,

Imidazoline Derivatives:
Ethylhexyl Dimethoxybenzylidene Dioxoimidazoline Propionate,

Benzalmalonate Derivatives:
Di-neopentyl 4′-methoxybenzalmalonate
Polyorganosiloxane with benzalmalonate functions as Polysilicone-15 sold under the commercial name PARSOL SLX by DSM NUTRITIONAL PRODUCTS

Derivatives of 4,4-Diarylbutadiene:
1,1-dicarboxy (2,2′-dimethyl-propyl)-4,4-diphenylbutadiene

Benzoxazole Derivatives:
2,4-bis-[5-1 (dimethylpropyl)benzoxazol-2-yl-(4-phenyl)-imino]-6-(2-ethylhexyl)-imino-1,3,5-triazine sold under the commercial name Uvasorb K2A by Sigma 3V and mixtures thereof.

Lipophilic Merocyanine Derivatives
-Octyl-5-N,N-diethylamino-2-phenysulfonyl-2,4-pentadienoate and mixtures thereof.

In some preferred embodiments, the sunscreen composition of the present invention comprises the UV protective agent which is selected from the group consisting of Ethylhexyl Methoxycinnamate (Uvinul® MC80), Ethylhexyl Triazone (Uvinul® T150), Methylene Bis-Benzotriazolyl Tetramethylbutylphenol (Tinosorb® M); Bis-Ethylhexyloxyphenol Methoxyphenyl Triazine (Tinosorb® S); Diethylamino Hydroxybenzoyl Hexyl Benzoate (Uvinul® A Plus Granular); Ethylhexyl Salicylate (Neo Heliopan OS).

The amount of the UV protective agent present in the sunscreen composition is from 5% to 50%, in particular, from 8% to 40%, more in particular from 10% to 20%, 25%, 30% or 35% by weight of the total composition.

Hydrophobically Modified Clay Mineral

The hydrophobically modified clay mineral is one of colloidal hydrous aluminum silicates having a tri-layer structure, and a substance derived by modification of a clay mineral represented by the following general formula (I) with a quaternary ammonium salt-type cationic surfactant can be used in the present invention:

(X,Y)₂₋₃(Si,Al)₄O₁₀(OH)₂Z_{1/3}·nH₂O

### Where X=Al, Fe(III), Mn(III), or Cr(III), Y=Mg, Fe(II), Ni, Zn or Li and Z=K, Na or Ca.

The quaternary ammonium salt-type cationic surfactant to be used here is represented by the following general formula (II):

N(R¹R²R³R⁴)X

Where R¹ denotes C₁₀₋₂₂ alkyl group or a benzyl group, R² denotes a methyl group or a C₁₀₋₂₂ alkyl group, R³ and R⁴ each denote a C₁₋₃ alkyl group or a hydroxyalkyl group; and X denotes a halogen atom or a methylsulfate residue.

Typical examples of the quaternary ammonium salt-type cationic compounds for the present invention include but are not limited to dodecyltrimethylammonium chloride, myristyltrimethylammonium chloride, cetyltrimethylammonium chloride, stearyltrimethylammonium chloride, arachyltrimethylammonium chloride, behenyltrimethylammonium chloride, myristyldimethylethylammonium chloride, cetyldimethylethylammonium chloride, stearyldimethylethylammonium chloride, arachyldimethylethylammonium chloride, behenyldimethylethylammonium chloride, myristyldiethylmethylammonium chloride, cetyldiethylmethylammonium chloride, stearyldiethylmethylammonium chloride, arachyldiethylmethylammonium chloride, behenyldiethylmethylammonium chloride, benzyldimethylmyristylammonium chloride, benzyldimethylcetylammonium chloride, benzyldimethylstearylammonium chloride, benzyldimethylbehenylammonium chloride, benzylmethylethylcetylammonium chloride, benzylmethylethylstearylammonium chloride, and dibehenyldihydroxyethylammonium chloride, and bromides corresponding to them, and dipalmitylpropylethylammonium methylsulfate.

The hydrophobically modified clays are known in the art. Typical examples are smectite clays such as hectorites, montmorillonites and bentonites, which have been made hydrophobic by treating them with an organic cationic compound. Typical examples of the hydrophobically modified clays are stearalkonium bentonite, dimethyldistearammonium hectorite, disteardimonium hectorite, dimethylalkylammonium hectorite, benzyldimethylstearylammonium hectorite, and magnesium aluminum silicate treated with distearyldimethylammonium chloride and preferably disteardimonium hectorite.

The amount of the hydrophobically modified clay present in the sunscreen composition is from 0.05% to 5%, in particular, from 0.1% to 1%, 1.5%, 2%, 2.5% or 3%, more in particular from 0.2% to 2% by weight of the total composition.

Oil Components

According to any one of the present embodiments, the suitable oil components include, but are not limited to, hydrophobic compounds such as vegetable oils, mineral oils (e.g., petrolatum), fatty esters (e.g., isopropyl palmitate, C₁₂-C₁₅ alkyl benzoate) including those fatty esters of glycerol and the like. It is desirable to include a combination of a “dry” emollient and an oily emollient. Dry emollients and oily emollients will be understood through a description of the material and examples suitable for such emollient. For example, dicaprylyl carbonate is considered a “dry” emollient, in that it dries and is removed from the skin of a user fairly rapidly. One particular oily emollient is heptyl undecylenate, which is oilier and is more difficult to remove from the skin of a user.

Suitable oils for the present invention include, but not are limited to: hydrocarbon-based oils of plant origin, such as liquid triglycerides of fatty acids containing from 4 to 10 carbon atoms, for instance heptanoic or octanoic acid triglycerides, or alternatively, for example, sunflower oil, corn oil, soybean oil, marrow oil, grapeseed oil, sesame seed oil, hazelnut oil, apricot oil, macadamia oil, arara oil, coriander oil, castor oil, avocado oil, caprylic/capric acid triglycerides, jojoba oil, shea butter oil and caprylyl glycol; synthetic esters and ethers, especially of fatty acids, for instance Purcellin oil, 2-octyldodecyl stearate, 2-octyldodecyl erucate, isostearyl isostearate; hydroxylated esters, for instance isostearyl lactate, octyl hydroxystearate, octyldodecyl hydroxystearate, diisostearyl malate or triisocetyl citrate; fatty alcohol heptanoates, octanoates or decanoates; polyol esters, for instance propylene glycol dioctanoate, neopentyl glycol diheptanoate and diethylene glycol diisononanoate; and pentaerythritol esters, for instance pentaerythrityl tetraisostearate, or isopropyl lauroyl sarcosinate, sold especially under the trade name Eldew SL 205 by the company Ajinomoto; linear or branched hydrocarbons, of mineral or synthetic origin, such as volatile or non-volatile liquid paraffins, and derivatives thereof, petroleum jelly, polydecenes, isohexadecane, isododecane, hydrogenated polyisobutene such as Parleam oil, or the mixture of n-undecane (C₁₁) and of n-tridecane (C₁₃) sold under the reference Cetiol® Ultimate by the company BASF; fluoro oils that are partially hydrocarbon-based and/or silicone-based, for instance those described in document JP-A-2 295 912; silicone oils, for instance volatile or non-volatile polydimethylsiloxanes (PDMS) with a linear or cyclic silicone chain, which are liquid or pasty at room temperature, in particular volatile silicone oils, especially cyclopolydimethylsiloxanes (cyclomethicones) such as cyclohexadimethylsiloxane and cyclopentadimethylsiloxane; polydimethylsiloxanes comprising alkyl, alkoxy or phenyl groups, which are pendent or at the end of a silicone chain, these groups containing from 2 to 24 carbon atoms; phenyl silicones, for instance phenyl trimethicones, phenyl dimethicones, phenyltrimethylsiloxydiphenylsiloxanes, diphenyl dimethicones, diphenylmethyldiphenyltrisiloxanes or 2-phenylethyl trimethylsiloxy silicates, and polymethylphenylsiloxanes; mixtures thereof.

Suitable oil components further include, for example, Guerbet alcohols based on fatty alcohols containing 6 to 18 and preferably 8 to 10 carbon atoms, esters of linear C₆₋₂₂ fatty acids with linear or branched C₆₋₂₂ fatty alcohols or esters of branched C₆₋₁₃ carboxylic acids with linear or branched C₆₋₂₂ fatty alcohols such as, for example, myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of linear C₆₋₂₂ fatty acids with branched alcohols, more particularly 2-ethyl hexanol, esters of C₁₈₋₃₈ alkylhydroxycarboxylic acids with linear or branched C₆₋₂₂ fatty alcohols, more especially Dioctyl Malate, esters of linear and/or branched fatty acids with polyhydric alcohols (for example propylene glycol, dimer diol or trimer triol) and/or Guerbet alcohols, triglycerides based on C₆₋₁₀ fatty acids, liquid mono-, di- and triglyceride mixtures based on C₆₋₁₈ fatty acids, esters of C₆₋₂₂ fatty alcohols and/or Guerbet alcohols with aromatic carboxylic acids, more particularly benzoic acid, esters of C₂₋₁₂ dicarboxylic acids with linear or branched alcohols containing 1 to 22 carbon atoms or polyols containing 2 to 10 carbon atoms and 2 to 6 hydroxyl groups, vegetable oils, branched primary alcohols, substituted cyclohexanes, linear and branched C₆₋₂₂ fatty alcohol carbonates, such as Dicaprylyl Carbonate (Cetiol® CC) for example, Guerbet carbonates based on C₆₋₁₈ and preferably C₅₋₁₀ fatty alcohols, esters of benzoic acid with linear and/or branched C₆₋₂₂ alcohols (for example Finsolv® TN), linear or branched, symmetrical or nonsymmetrical dialkyl ethers containing 6 to 22 carbon atoms per alkyl group, such as Dicaprylyl Ether (Cetiol® OE) for example, ring opening products of epoxidized fatty acid esters with polyols, silicone oils (cyclomethicone, silicon methicone types, etc.) and/or aliphatic or naphthenic hydrocarbons such as, for example, squalane, squalene or dialkyl cyclohexanes.

Additional examples include benzoic acid esters of C₉-C₁₅ alcohols, isononyl iso-nonanoate, C₁₂-C₁₅ alkyl benzoate, or any combinations thereof.

Specific examples of oils and/or emollients include cocoglyceride, cyclomethicone, dimethicone, dicapryl maleate, caprylic/capric triglyceride, isopropyl myristate, octyl stearate, isostearyl linoleate, lanolin oil, coconut oil, cocoa butter, olive oil, avocado oil, aloe extracts, jojoba oil, castor oil, fatty acid, oleic acid, stearic acid, fatty alcohols, cetyl alcohol, hexadecyl alcohol, diisopropyl adipate, hydroxybenzoate esters, benzoic acid esters of C₉-C₁₅ alcohols, isononyl isononanoate, alkanes, mineral oil, silicone, dimethylpolysiloxane, ether, polyoxypropylene butyl ether, polyoxypropylene cetyl ether, C₁₂-C₁₅ alkyl benzoate, aryl alkyl benzoate, Isopropyl Lauroyl sarcosinate, and any combinations thereof.

In some preferred embodiments, a useful oil component for the present invention includes dry emollients. Any suitable dry emollients may be used, including for example, a carbonate ester, and ester of carbonic acid. A preferred carbonate ester is dicapryl carbonate, also known as dioctylcarbonate, dicaprylyl carbonate, or carbonic acid, dioctyl ester. Dicaprylyl carbonate is commercially available under the trade name Cetiol® CC from BASF. Other useful carbonate esters include, but not are limited to cetyl carbonate, cetearyl carbonate, stearyl carbonate, C₁₂-C₁₅ alkyl carbonate and the like, the dry emollient is particularly useful in the present inventive composition.

The amount of the oil components present in the sunscreen composition is from 1% to 60%, in particular, from 5% to 10%, 15%, 20%, 25%, 30%, 35% or 40%, more in particular from 10% to 20%, 25%, 30% or 35% by weight of the total composition.

Associative Polymer

The composition of the present invention comprises at least one associative polymer. As used herein, the term “associative” polymer is defined as a water-soluble or water-swellable polymer that has chemically attached groups that are capable of hydrophobic associations similar to those of conventional surfactants. As used herein, the attached “hydrophobic or hydrophobic group” is any chemical group that promotes water insolubility. The associative polymer used in the present cosmetic composition, apart from their thickening effect, offer an advantageous application as film former starting from the aqueous medium.

In some preferred embodiments, the silicone-based hydrophobic polymer may be selected from dimethicone, amodimethicone, dimethiconol, silicone polyurethane silicone acrylate or a combination thereof. Specifically, the polymer may be a silicone acrylate copolymer, such as is a copolymer comprising a poly(alkyl)acrylate backbone and a dimethicone copolymer grafted to an acrylate ester side chain. By silicone based is meant that the hydrophobic polymer comprises at least one silicone moiety. Particular mention may be made of the silicone acrylate copolymers, in particular copolymer comprising a poly(alkyl)acrylate backbone and a dimethicone polymer grafted to an alkyl ester side chain, such as commercially available cyclopentasiloxyna (and) Acrylates/polytrimethylsiloxymethacrylate copolymer under tradename FA 4001 CM from Dow Corning, Isododecane (and) Acrylates/Polytrimethylsiloxymethacrylate copolymer under tradename FA 4002 ID from Dow Corning, Dimethicone (and) Acrylates/polytrimethylsiloxymethacrylate copolymer under tradename FA 4003 DM from Dow Corning, Isodedecane (and) Acrylates/polytrimethylsiloxymethacrylate copolymer under tradename FA 4004 ID from Dow Corning, Acrylates/polytrimethylsiloxymethacrylate copolymer (and) Laureth-1 phosphate under tradename FA 4103 from Dow Corning, and Isododecane (and) Trimethylsiloxysilicate/Dimethiconol crosspolymer under tradename FC-5002 IDD from Dow Corning.

In some preferred embodiments, the associative polymer comprises a copolymer of (meth)acrylic acid ester of an ethoxylated C₈-C₃₀, preferably C₁₂-C₃₀, in particular C₁₆-C₂₂, more in particular C₁₈-C₂₂ fatty alcohol and one or more monomers of acrylic acid, methacrylic acid or one of their simple esters; In some preferred embodiments, the associative polymer comprises a copolymer of itaconic acid ester of ethoxylated C₈-C₃₀, preferably C₁₂-C₃₀, in particular C₁₆-C₂₂, more in particular C₁₈-C₂₂ fatty alcohol and one or more monomers of acrylic acid, methacrylic acid or one of their simple ester. In some embodiments, the associative polymer comprises a copolymer of (meth)acrylic acid ester of ethoxylated C₈-C₃₀, preferably C₁₂-C₃₀, in particular C₁₆-C₂₂, more in particular C₁₈-C₂₂ fatty alcohols and one or more monomers of acrylic acid, methacrylic acid or one of their simple esters, or a copolymer of itaconic acid ester of ethoxylated C₈-C₃₀, preferably C₁₂-C₃₀, in particular C₁₆-C₂₂, more in particular C₁₈-C₂₂ fatty alcohol and one or more monomers of acrylic acid, methacrylic acid or one of their simple ester, wherein the ethoxylated fatty alcohols in each case comprise 5 to 35, in particular 7 to 30, more in particular 7 to 25 ethylene oxide (EO) radicals. Non-limiting example of those polymers include Tinovis® GTC (INCI: Acrylates/Beheneth-25 Methacrylate Copolymer); Aculyn® 22 (INCI: Acrylates/Steareth-20 Methacrylate Copolymer); Aculyn® 88 (INCI: Acrylates/Steareth-20 Methacrylate Crosspolymer; Acrylated/Steareth-20 Itaconate copolymer; Acrylate/Ceteth-20 Itaconate copolymer and Acrylates/Palmeth25).

In some preferred embodiments, it is likewise possible with advantage to use the associative polymer based on acrylamido methylpropanesulfonic acid (AMPS) and at least one ethylenically unsaturated monomer comprising at least one hydrophobic part containing from 8 to 30 carbon atoms, in particular from 10 to 30 carbon atoms and more particular from 12 to 22 carbon atoms, in addition, the hydrophobic radical may also comprise at least one alkylene oxide unit and in particular a polyoxyalkylene chain. The polyoxyalkylene chain may be formed from ethylene oxide units and/or propylene oxide units and even more particular be formed solely from ethylene oxide units, preferably, the at least one ethylenically unsaturated monomer is acrylic or methacrylic ester of ethoxylated alcohols RO—(CH₂CH₂O)ₘH, in which R is an alkyl radical having 12 to 30 carbon atoms and m is a number from 3 to 35, and CH₂═CH—COO—(CH₂CH₂—COO)ₙX in which n is a number from 0 to 10 and X is a counterion and is preferably H⁺, Na⁺ and/or NH₄⁺. In the case of the copolymers, preferably one or more structural units based on the following comonomers are present in the copolymers: acrylic acid, methacrylic acid, acrylamide, dimethyl-acrylamide, vinylpyrrolidone (VP), hydroxyethyl acrylate, and hydroxyethyl methacrylate. More particularly, the suitable associative polymer can be copolymer of AMPS and (meth)acrylic ester of ethoxylated C₈-C₃₀, or C₁₀-C₃₀, or C₁₂-C₂₂, or C₁₂-C₁₈ fatty alcohol. In some embodiments, the suitable associative polymer can be copolymer of AMPS and (meth)acrylic ester of ethoxylated C₈-C₃₀, or C₁₂-C₃₀, or C₁₂-C₂₂, or C₁₂-C₁₈ fatty alcohol, wherein the ethoxylated fatty alcohol in each case comprise 5 to 35, in particular 7 to 30, more in particular 7 to 25 ethylene oxide (EO) radicals. Suitable AMPS copolymers are, for example, ammonium acryloyldimethyltaurate/beheneth-25-methacrylate copolymer such as Aristoflex® HMB from Clariant, ammonium acryloyldimethyltaurate/beheneth-25 methacrylate crosspolymer such as Aristoflex® BLV from Clariant, ammonium Acryloyldimethyltaurate/Steareth-25 Methacrylate crosspolymer such as Aristoflex HMS® by Clariant, ammonium acryloyldimethyltaurate/steareth-8 methacrylate copolymer such as Aristoflex® SNC, Ammonium acryloyldimethyltaurate/laureth-7 methacrylate copolymer such as Aristoflex® LNC.

The amount of the associative polymers present in the sunscreen composition is from about 0.1% to 4%, in particular from about 0.2% to 1%, 1.5%, 2%, 2.5%, 3% or 3.5%, and more in particular from about 0.2% to 1% by weight of the total composition.

Emulsifiers

The composition of the present invention may also include an emulsifier in an amount between about 0 to about 10%, preferably about 0 to 1%, 1.5%, 2%, 2.5%, 3%, 3.5%, 4%, 4.5%, 5% or 5.5%, 6%, 6.5%, 7%, 7.5% or 8%, more preferably about 0 to 5% by weight of the total composition. Suitable emulsifiers can be any of a wide variety capable of facilitating an emulsion. Anionic or nonionic emulsifiers are preferred. Suitable emulsifier types include soaps; alkyl, aryl or alkylaryl, or acyl-modified versions of the following moieties: sulfates, ether sulfates, monoglyceryl ether sulfates, sulfonates, sulfosuccinates, ether sulfosuccinates, sulfosuccinamates, amidosulfosuccinates, carboxylates, amidoethercarboxylates, succinates, sarcosinates, amino acids, taurates, sulfoacetates, and phosphates; polyoxyethylene fatty ether phosphates, acyl lactylates, and mixture thereof. Notable anionic emulsifiers are phosphate esters, such as cetyl phosphate salts, such as potassium cetyl phosphate. In some embodiments, the composition may further comprise nonionic, amphoteric or polymeric emulsifiers. Examples of suitable chemical classes of nonionic surfactant include ethoxylates of amide, fatty acid amides, ethoxylated fatty acids, ethoxylated esters, ethoxylated ethers, ethoxylated alcohols, polyoxyethylene derivatives of polyol ester, noncrosslinked silicone copolymers such as alkoxy or alkyl dimethiocone copolyols, silicones having pendant hydrophilic moieties such as linear silicones having pendant polyether groups or polyglycerol groups, and crosslinked elastomeric solid organopolysiloxanes comprising at least one hydrophilic moiety. Specific nonionic emulsifiers include, for example, addition of 2 to 30 mol ethylene oxide and/or 0 to 5 mol propylene oxide onto linear C₈₋₂₂ fatty alcohols, onto C₁₂₋₂₂ fatty acids, onto alkyl phenols containing 8 to 15 carbon atoms in the alkyl group and onto alkylamines containing 8 to 22 carbon atoms in the alkyl group; alkyl oligoglycosides containing 8 to 22 carbon atoms in the alkyl group and ethoxylated analogs thereof; addition products of 1 to 15 mol ethylene oxide onto castor oil and/or hydrogenated castor oil; addition products of 15 to 60 mol ethylene oxide onto castor oil and/or hydrogenated castor oil; partial esters of glycerol and/or sorbitan with unsaturated, linear or saturated, branched fatty acids containing 12 to 22 carbon atoms and/or hydroxycarboxylic acids containing 3 to 18 carbon atoms and addition products thereof onto 1 to 30 mol ethylene oxide; partial esters of polyglycerol (average degree of self-condensation 2 to 8), polyethylene glycol (molecular weight 400 to 5,000), trimethylolpropane, pentaerythritol, sugar alcohols (for example sorbitol), alkyl glucosides (for example methyl glucoside, butyl glucoside, lauryl glucoside, other C₆-C₂₂ alkyl glucoside) and polyglucosides (for example cellulose) with saturated and/or unsaturated, linear or branched fatty acids containing 12 to 22 carbon atoms and/or hydroxycarboxylic acids containing 3 to 18 carbon atoms and addition products thereof onto 1 to mol ethylene oxide; mixed esters of pentaerythritol, fatty acids, citric acid and fatty alcohol and/or mixed esters of fatty acids containing 6 to 22 carbon atoms, methyl glucose and polyols, preferably glycerol or polyglycerol, mono-, di- and trialkyl phosphates and mono-di- and/or tri-PEG-alkyl phosphates and salts thereof, wool wax alcohols, polysiloxane/polyalkyl/polyether copolymers and corresponding derivatives, block copolymers, for example Polyethyleneglycol-30 Dipolyhydroxystearate; polymer emulsifiers, for example Pemulen types (TR-1, TR-2) of Goodrich, polyalkylene glycols and glycerol carbonate. The addition products of ethylene oxide and/or propylene oxide onto fatty alcohols, fatty acids, alkylphenols or onto castor oil are known commercially available products. They are homolog mixtures of which the average degree of alkoxylation corresponds to the ratio between the quantities of ethylene oxide and/or propylene oxide and substrate with which the addition reaction is carried out. C₁₂₁₁₈ fatty acid monoesters and diesters of addition products of ethylene oxide onto glycerol are known as refatting agents for cosmetic formulations. Sorbitan ester emulsifiers including sorbitan monoisostearate, sorbitan sesquiisostearate, sorbitan diisostearate, sorbitan triisostearate, sorbitan monooleate, sorbitan sesquioleate, sorbitan dioleate, sorbitan trioleate, sorbitan monoerucate, sorbitan sesquierucate, sorbitan dierucate, sorbitan trierucate, sorbitan monoricinoleate, sorbitan sesquiricinoleate, sorbitan diricinoleate, sorbitan triricinoleate, sorbitan monohydroxystearate, sorbitan sesquihydroxystearate, sorbitan dihydroxystearate, sorbitan trihydroxystearate, sorbitan monotartrate, sorbitan sesquitartrate, sorbitan ditartrate, sorbitan tritartrate, sorbitan monocitrate, sorbitan sesquicitrate, sorbitan dicitrate, sorbitan tricitrate, sorbitan monomaleate, sorbitan sesquimaleate, sorbitan dimaleate, sorbitan trimaleate and technical mixtures thereof. Addition products of 1 to 30 and preferably 5 to 10 mol ethylene oxide onto the sorbitan esters mentioned are also suitable. polyglycerol esters including Polyglyceryl-2 Dipolyhydroxystearate (Dehymuls® PGPH), Polyglycerin-3-Diisostearate (Lameform® TGI), Polyglyceryl-4 Isostearate (Isolan® GI 34), Polyglyceryl-3 Oleate, Diisostearoyl Polyglyceryl-3 Diisostearate (Isolan® PDI), Polyglyceryl-3 Methylglucose Distearate (Tego Care® 450), Polyglyceryl-3 Beeswax (Cera Belling®), Polyglyceryl-4 Caprate (Polyglycerol Caprate T2010/90), Polyglyceryl-3 Cetyl Ether (Chimexane®) NL), Polyglyceryl-3 Distearate (Cremophor® GS 32) and Polyglyceryl Polyricinoleate (Admul® WOL 1403), Polyglyceryl Dimerate Isostearate and mixtures thereof. Examples of other suitable polyolesters are the mono-, di- and triesters of trimethylolpropane or pentaerythritol with lauric acid, coco fatty acid, tallow fatty acid, palmitic acid, stearic acid, oleic acid, behenic acid and the like optionally reacted with 1 to 30 mol ethylene oxide.

Examples of suitable chemical classes of amphoteric emulsifiers include alkyl betaines, amidoalkyl betaines, alkylamphoacetates, amidoalkyl sultaines, amphophosphates, phosphorylated imidazolines, carboxyalkyl alkyl polyamines, alkylamino-dipropionates, alkylamphoglycinates (mono or di); alkylamphoproprionates, N-alkyl b-aminoproprionic acids, and alkylpolyamino carboxylates. Suitable amphoteric surfactants include, for example, the N-alkyl-N,N-dimethyl ammonium glycinates, for example cocoalkyl dimethyl ammonium glycinate, N-acylaminopropyl-N,N-dimethyl ammonium glycinates, for example cocoacylaminopropyl dimethyl ammonium glycinate, and 2-alkyl-3-carboxymethyl-3-hydroxyethyl imidazolines containing 8 to 18 carbon atoms in the alkyl or acyl group and cocoacylaminoethyl hydroxyethyl carboxymethyl glycinate. N-alkyl glycines, N-alkyl propionic acids, N-alkylaminobutyric acids, N-alkyliminodipropionic acids, N-hydroxyethyl-N-alkylamidopropyl glycines, N-alkyl taurines, N-alkyl sarcosines, 2-alkylaminopropionic acids and alkylaminoacetic acids containing around 8 to 18 carbon atoms in the alkyl group. Particularly preferred ampholytic surfactants are N-coco-alkylaminopropionate, cocoacylaminoethyl aminopropionate and C_{12/18} acyl sarcosine.

In some embodiments, the preferred emulsifiers used in the present invention includes polyglyceryl-2 dipolyhydroxystearate (Dehymuls® PGPH), cetyl PEG/PPG-10/1 dimethicone, PEG-10 dimethicone, lauryl PEG/PPG-18/18 methicone.

In some preferred embodiment, the sunscreen compositions are emulsifier-free compositions which does not contain any emulsifier. Conventional emulsifiers have been found to be potentially irritating to the skin and therefore undesirable. Emulsifier-free cosmetic composition of the prior art in general have the disadvantage that they are either unstable, have a narrow range of use or are limited to a limited choice of starting substances. It is usually furthermore the case that compositions of the prior art contain little or even no oils or emollients. Another objective of the present invention is to prepare emulsifier-free sunscreen composition which do not have the disadvantages of the prior art and provide a satisfied sensory experience. Surprisingly and unexpectedly, the present inventive sunscreen compositions have proved to be a promising approach for an emulsifier-free sunscreen cosmetic with the goal of obtaining sufficiently stable and cosmetically attractive products, which help to avoid the disadvantage connected with conventional emulsifiers.

Polymer Powder

The sunscreen compositions of the present invention may further comprise the polymer powders which render the composition silky and powdery skin feel. Example of the polymer powders include dimethicone crosspolymer, dimethicone/vinyl dimethicone crosspolymer, polymethylsilsesquioxane, polyethylene and methyl polymetharcrylate.

Benefit Agent

The term “benefit agent” as used herein should be broadly understood and intends an agent which when included in a personal washing composition in an appropriate amount is intended to produce a hair and/or skin care benefit.

In addition to the aforementioned ingredients, other ingredients usually used in cosmetic can be added as necessary into the sunscreen cosmetics of the present invention is not adversely affected. The composition of the present invention may further comprise one or more benefit agent, such as starch, emollients; moisturizers; skin conditioners; waxes; alcohols; liquid fats and oils; anti-acne agents, such as tretinoin, isotretinoin, motretinide, adapalene, tazarotene azelaic acid; anti-inflammatory agents, such as ibuprofenm naproxen, hetprofen, botanical extract such as alnus, arnica, artemisia capillaris, asiasarum root, calendula, chamomile; refreshing agents; nourishing agents; anti-wrinkle agents, fragrances, skin-coloring agents.

The sunscreen composition according to the present invention may optionally further comprise other ingredients such as, for example, preservatives such as benzyl alcohol, methyl parabenm, propyl paraben, imidazolidinyl urea, sodium benzoate, potassium sorbate, salicyclic acid, methylchloroisothiazolinone and methylisothiazolinone; pH adjusting agent such as citric acid, succinic acid, phosphoric acid, sodium hydroxide, and sodium carbonate; Any other benefit ingredients may be added to the sunscreen composition depending on the desired properties of the sunscreen compositions.

In one specific embodiment, the sunscreen composition according to the present invention further comprises a fragrance material or perfume.

According to any one of the invention embodiments, the sunscreen composition further comprises a carrier, or a mixture of such carriers, which are suitable for application to the skin and/or hair. Suitable carriers for use with skin compositions include water, C₁-C₆ alcohols, lower alkyl acetate and mixtures thereof. In some embodiments, the carriers can also contain a wide variety of additional materials such as acetone, hydrocarbons such as isobutane, hexane, decene, halogenated hydrocarbons and volatile silicones such as cyclomethicone. The carriers are present in the composition ranging from about 0.5% to about 99%, preferably from about 5% to about 99%, more preferably from about 10% to about 98%, based on the total weight of the composition.

The composition of the present invention comprises a cosmetically acceptable base. The cosmetically acceptable bases are such as to have a product in preferably a cream, lotion, gel or emulsion format.

The term “cosmetically acceptable” refers to ingredients typically used in personal care compositions and is intended to underscore that materials that are toxic when present in the amounts typically found in personal care compositions are not contemplated as part of the present invention.

According to any one embodiment of the present invention, the invention relates to a sunscreen composition comprising:
a) from 5 to 50% by weight of at least one UV protective agent;
b) from 0.05 to 5% by weight of at least one hydrophobically modified clay mineral;
c) from 1 to 60% by weight of at least one oil component;
d) from 0.1 to 4% by weight of at least one associative polymer,
   wherein the UV protective agent is selected from the group consisting of ethylhexyl methoxycinnamate, ethylhexyl Triazone, methylene bis-benzotriazolyl tetramethylbutylphenol, bis-ethylhexyloxyphenol methoxyphenyl triazine, diethylamino hydroxybenzoyl hexyl benzoate, ethylhexyl salicylate and a combination thereof; wherein the associative polymer is selected from the group consisting of a copolymer of (meth)acrylic acid ester of ethoxylated C₈-C₃₀, preferably C₁₂-C₃₀, in particular C₁₆-C₂₂, more in particular C₁₈-C₂₂ fatty alcohol and one or more monomers of acrylic acid, methacrylic acid or one of their simple ester; a copolymer of itaconic acid ester of ethoxylated C₈-C₃₀, preferably C₁₂-C₃₀, in particular C₁₆-C₂₂, more in particular C₁₈-C₂₂ fatty alcohol and one or more monomers of acrylic acid, methacrylic acid or one of their simple ester; and a copolymer based on acrylamidomethylpropanesulphonic acid and (meth)acrylic ester of ethoxylated C₈-C₃₀, or C₁₀-C₃₀, or C₁₂-C₂₂, or C₁₂-C₁₈ fatty alcohol; wherein the ethoxylated fatty alcohol in each case comprise 5 to 35, in particular 7 to 30, more in particular 7 to 25 ethylene oxide radicals; wherein the hydrophobically modifed clay mineral is selected from the group consisting of stearalkonium bentonite, dimethyldistearammonium hectorite, disteardimonium hectorite, dimethylalkylammonium hectorite, benzyldimethylstearylammonium hectorite, magnesium aluminum silicate treated with distearyldimethylammonium chloride and a combination thereof; preferably disteardimonium hectorite; wherein the oil component is selected from the group consisting of dicapryl carbonate, cetyl carbonate, cetearyl carbonate, stearyl carbonate and a mixture thereof, preferably dicapryl carbonate.

According to any one embodiment of the present invention, the invention relates to a sunscreen composition comprising:
a) from 5 to 50% by weight of at least one UV protective agent;
b) from 0.05 to 5% by weight of at least one hydrophobically modified clay mineral;
c) from 1 to 60% by weight of at least one oil component;
d) from 0.1 to 4% by weight of at least one associative polymer,
wherein the UV protective agent is selected from the group consisting of ethylhexyl methoxycinnamate, ethylhexyl Triazone, methylene bis-benzotriazolyl tetramethylbutylphenol, bis-ethylhexyloxyphenol methoxyphenyl triazine, diethylamino hydroxybenzoyl hexyl benzoate, ethylhexyl salicylate and a combination thereof; wherein the associative polymer is selected from the group consisting of acrylates/beheneth-25 methacrylate copolymer, acrylates/steareth-methacrylate copolymer, acrylates/steareth-20 methacrylate crosspolymer, acrylated/steareth-20 itaconate copolymer; acrylate/ceteth-20 itaconate copolymer and acrylates/palmeth-25 itaconate copolymer, ammonium acryloyldimethyltaurate/beheneth-25-methacrylate copolymer, ammonium acryloyldimethyltaurate/beheneth-25 methacrylate crosspolymer, ammonium acryloyldimethyltaurate/Steareth-25 methacrylate crosspolymer, ammonium acryloyldimethyltaurate/steareth-8 methacrylate copolymer, ammonium acryloyldimethyltaurate/laureth-7 methacrylate copolyme and a combination thereof; wherein the hydrophobically modifed clay mineral is selected from the group consisting of stearalkonium bentonite, dimethyldistearammonium hectorite, disteardimonium hectorite, dimethylalkylammonium hectorite, benzyldimethylstearylammonium hectorite, magnesium aluminum silicate treated with distearyldimethylammonium chloride and a combination thereof, preferably disteardimonium hectorite; wherein the oil component is selected from the group consisting of dicapryl carbonate, cetyl carbonate, cetearyl carbonate, stearyl carbonate and a mixture thereof, preferably dicapryl carbonate.

Personal care compositions are usually prepared by mixing individual components using any conventional blending technique known in the prior art such as conventional stirring, shaking or tumbling. These components may be supplied as concentrated solutions which are diluted and/or and combined in appropriate ratios by the skilled person. The invention covers any concentrate to be used as component ingredient to prepare a composition of the invention, and especially to concentrates containing limited levels of water due to some reasons from a cost and environmental perspective.

### EXAMPLES

Materials:

TABLE 1

| Name | INCI Name |
|---|---|
| Uvinul ® MC 80 plus | Ethylhexyl Methoxycinnamate |
| Uvinul ® A plus | Diethylamino Hydroxybenzoyl Hexyl Benzoate |
| Uvinul ® T150 | Ethylhexyl Triazone |
| Tinosorb ® S | Bis-Ethylhexyloxyphenol Methoxyphenyl |
| | Triazine |
| Neo Heliopan ® OS | Ethylhexyl Salicylate |
| Z-cote ® HP 1 | Silicone coated Zinc Oxide |
| Cetiol ® CC | Stearalkonium Hectorite |
| Benton 38V CG from | Disteardimonium Hectorite |
| Elementis | |
| Tinovis ® GTC UP | Acrylates/Beheneth-25 Methacrylate |
| | Copolymer |
| Aristoflex ® BLV | Ammonium acryloyldimethyltaurate/beheneth- |
| | 25 methacrylate crosspolymer |
| Hostacerin ® AMPS | Ammonium polyacryloyl dimethyl taurate |
| Cosmedia ® SP | Sodium Polyacrylate |
| Plantacare ® 2000 UP | Decyl glucoside |
| Belsil DM 1 Plus | Dimethicone |
| SILICA BEAD SB-300 | Silica |
| Gransil PSQ | Polymethylsilsesquioxane |

Measurements/Methods

Film Application and Preparation

Apply 6 g sunscreen formulation on the PMMA board by draw down device to get uniform film of thickness of 0.076 mm wet film. The PMMA board is then moved in an oven heated at 38° C. for at least 30 minutes to mimic the film formation under skin temperature. The film was taken out from the oven after the preparation.

Film Morphology, Roughness and Thickness Evaluation

The film was then evaluated by white light interferometry (WLI, BRUKER Contour GT-K) which was used to obtain the morphological information including roughness and thickness of the films.

Roughness=\sqrt{AvgΔ²}

(unit: nm) (Δ means the distance from each point on the film surface to the middle layer)

The roughness value is the average of the roughness parameters measured three times at each of the three randomly chosen sites on each film.

The surface roughness of the films was graded as below:
less rough++(Δ<700 nm)
medium rough+(Δ=700 to 3000 nm)
rougher−(Δ>3000 nm)

The initial film thickness was measured when the film is made, and the thickness measurement was then made 30 mins later at room temperature. The film thickness variation Δd was measured and calculated as below:

Δd=(film thickness measured at room temperature−initial film thickness)/initial film thickness

The film thickness may be measured by methods known to one of skill in the art, for example, by the combination of calipers and a calibrated microscope. The thickness of the film may also be digitally measured from a micrograph of the film cross-section.

In comparison to the original thickness applied, the film thickness variation is classified into three groups:
minor change++(Δd 15%)
medium change+(Δd 40%)
major change−(Δd>40%)

Water-Resistance Evaluation

0.03 g of sunscreen formulation was applied on the 5*5 cm PMMA board. The PMMA board was placed in the climate-control room at temperature of 23° C. and humidity of 60% for 30 minutes. The PMMA board was then held on a silica gel board with black background and a spray bottle filled with tap water was used to spray the water 4 times to the surface of the PMMA board at the distance of 10 cm. Pictures were taken (by Canon 6D Mark II) at T₁ (before using spray water) and at T₂ (after using spray water) to record the water-drop mixed with melted sunscreen product. The pictures were then analyzed by picture analysis software ImageJ to calculate the changing of shaded area (A Area %):

Δ Area %=1−(T₁−T₂)/T₁.

The smaller changing percent illustrates better water-resistance performance. The water-resistance results were classified into three different categories:
Strong water-resistance++(Δ Area %≤15%)
Medium water-resistance+(Δ Area %≤0.40%)
Weak water-resistance−(Δ Area %>40%)

Film Adhesion for Anti-Dust Effect Evaluation

4 g of sunscreen formulation was applied on the PMMA board and then switched on a draw down device with draw down bar: 3 mil wet film. 2 g more sunscreen formulation was added in order to have a uniform film. The PMMA board was then placed in the in the oven at 38° C. for 30 minutes. 2 g of standard sand (sigma-Aldrich, 5 μm) was weighted and put into a petri dish and the PMMA board coated with film was placed onto the petri dish and the film was capable of contacting with the sand. The PMMA board and the petri dish was turned over and shaken gently for 30 seconds to make sure all the area of the film is covered by the sand. The PMMA board was then removed and the remaining sand in the petri dish was measured and weighted.

Adhesion ratio %=((initial sand weight in petri dish−remaining sand weight in petri dish)/initial sand weight in petri dish)*100%

The lower the adhesion ratio % is the better the anti-dust effect is. The anti-dust effect was graded into three categories:
high anti-dust effect++(adhesion ratio %=from 10% to 30%)
medium anti-dust effect+(adhesion ratio %=from 30% to 50%)
low anti-dust effect−(adhesion ratio %>50%

Formulations

TABLE 2
Formulas of the inventive examples

| Ingredients | Ex 1 | Ex 2 | Ex 3 | Ex 4 | Ex 5 |
|---|---|---|---|---|---|
| Ethylhexyl Methoxycinnamate | 10% | 10% | 10% | 10% | 10% |
| Diethylamino Hydroxybenzoyl | 5.5% | 5.5% | 5.5% | 5.5% | 5.5% |
| Hexyl Benzoate | | | | | |
| Ethylhexyl Triazone | 1.0% | 1.0% | 1.0% | 1.0% | 1.0% |
| Bis-Ethylhexyloxyphenol | 1.0% | 1.0% | 1.0% | 1.0% | 1.0% |
| Methoxyphenyl Triazine | | | | | |
| Ethylhexyl Salicylate | — | — | — | 4.0% | 4.0% |
| Silicone coated Zinc Oxide | 6.0% | 6.0% | 6.0% | 6.0% | 6.0% |
| Dicaprylyl Carbonate | 5.0% | 5.0% | 5.0% | 5.0% | 5.0% |
| Decyl glucoside | | | | — | — |
| Stearalkonium Hectorite | 0.5% | 1.0% | 0.1% | 0.5% | — |
| Disteardimonium hectorite | — | — | — | — | 0.5% |
| Butylene Glycol | 2.0% | 2.0% | 2.0% | 2.0% | 2.0% |
| Acrylates/Beheneth-25 | 3.0% | 0.5% | 3.0% | — | 1.5% |
| Methacrylate Copolymer | | | | | |
| Ammonium | — | — | — | 0.5% | — |
| acryloyldimethyltaurate/beheneth- | | | | | |
| 25 methacrylate crosspolymer | | | | | |
| Sodium Polyacrylate | — | — | — | — | — |
| Sodium Chloride | 1.0% | 0% | 2.0% | 1.0% | 1.0% |
| Dimethicone | 10% | 10% | 10% | 10% | 10% |
| Silica | 2.0% | 2.0% | 2.0% | 2.0% | 2.0% |
| Polymethylsilsesquioxane | 3.0% | 3.0% | 3.0% | 3.0% | 3.0% |
| Water | Up to | Up to | Up to | Up to | Up to |
| | 100% | 100% | 100% | 100% | 100% |

* Ex 1 to Ex 5 are the inventive examples 1 to 5.
* All the percentages refer to weight percent of active content.

TABLE 3
Formulas of the comparative examples

| | Comp | Comp | Comp | Comp | Comp | Comp | Comp |
|---|---|---|---|---|---|---|---|
| Ingredients | Ex 1 | Ex 2 | Ex 3 | Ex 4 | Ex 5 | Ex 6 | Ex 7 |
| Ethylhexyl Methoxycinnamate | 10% | 10% | 10% | 10% | 10% | 10% | 10% |
| Diethylamino Hydroxybenzoyl | 5.5% | 5.5% | 5.5% | 5.5% | 5.5% | 5.5% | 5.5% |
| Hexyl Benzoate | | | | | | | |
| Ethylhexyl Triazone | 1.0% | 1.0% | 1.0% | 1.0% | 1.0% | 1.0% | 1.0% |
| Bis-Ethylhexyloxyphenol | 1.0% | 1.0% | 1.0% | 1.0% | 1.0% | 1.0% | 1.0% |
| Methoxyphenyl Triazine | | | | | | | |
| Ethylhexyl Salicylate | — | — | — | 4.0% | 4.0% | 4.0% | 4.0% |
| Silicone coated Zinc Oxide | 6.0% | 6.0% | 6.0% | 6.0% | 6.0% | 6.0% | 6.0% |
| Dicaprylyl Carbonate | 5.0% | 5.0% | 5.0% | 5.0% | 5.0% | 5.0% | 5.0% |
| Decyl glucoside | — | — | — | — | — | — | 0.5% |
| Stearalkonium Hectorite | 0.5% | — | — | 0.5% | 0.5% | 0.5% | — |
| Disteardimonium hectorite | — | — | — | — | — | — | — |
| Butylene Glycol | 2.0% | 2.0% | 2.0% | 2.0% | 2.0% | 2.0% | 2.0% |
| Acrylates/Beheneth-25 | — | 1.5% | — | — | — | — | — |
| Methacrylate | | | | | | | |
| Copolymer | | | | | | | |
| Ammonium | — | — | — | — | — | — | — |
| acryloyldimethyltaurate/beheneth- | | | | | | | |
| 25 methacrylate crosspolymer | | | | | | | |
| Ammonium polyacryloyl dimethyl | — | — | — | 0.5% | — | — | — |
| taurate | | | | | | | |
| Sodium Polyacrylate | — | — | — | — | — | 0.5% | — |
| Sodium Chloride | 1.0% | 1.0% | 1.0% | 1.0% | 1.0% | 1.0% | 1.0% |
| Dimethicone | 10% | 10% | 10% | 10% | 10% | 10% | 10% |
| Silica | 2.0% | 2.0% | 2.0% | 2.0% | 2.0% | 2.0% | 2.0% |
| Polymethylsilsesquioxane | 3.0% | 3.0% | 3.0% | 3.0% | 3.0% | 3.0% | 3.0% |
| Water | Up to | Up to | Up to | Up to | Up to | Up to | Up to |
| | 100% | 100% | 100% | 100% | 100% | 100% | 100% |

* Comp Ex 1 to Comp Ex 7 are the comparative examples 1 to 7.
* All the percentages refer to weight percent of active content.

Evaluation Results

TABLE 4
Test results of the inventive examples

| Evaluation | Ex 1 | Ex 2 | Ex 3 | Ex 4 | Ex 5 |
|---|---|---|---|---|---|
| Roughness | ++ | ++ | ++ | n.d. | n.d. |
| Thickness variation | ++ | ++ | ++ | ++ | ++ |
| Water resistance (spray water | ++ | ++ | ++ | ++ | ++ |
| at room temperature) | | | | | |
| Water resistance (spray water | ++ | ++ | ++ | n.d. | n.d. |
| at 38° C.) | | | | | |
| Anti-dust performance | ++ | ++ | ++ | n.d. | n.d. |

* n.d.: not determined

TABLE 5
Test results of the comparative examples

| | Comp | Comp | Comp | Comp | Comp | Comp | Comp |
|---|---|---|---|---|---|---|---|
| Evaluation | Ex 1 | Ex 2 | Ex 3 | Ex 4 | Ex 5 | Ex 6 | Ex 7 |
| Roughness | − | + | − | n.d. | n.d. | n.d. | n.d. |
| Thickness variation | + | − | − | + | + | + | − |
| Water resistance (spray | + | + | − | − | − | − | − |
| water at room temperature) | | | | | | | |
| Water resistance (spray | + | + | − | n.d. | n.d. | n.d. | n.d. |
| water at 38° C.) | | | | | | | |
| Anti-dust performance | + | − | − | n.d. | n.d. | n.d. | n.d. |

* n.d.: not determined

The test results illustrate that the inventive examples comprising at least one UV protective agent, one hydrophobically modified clay mineral, one oil component and one associative polymer, bring about advantageous technical effects in film morphology, water resistance, and anti-dust performance.

1.-11. (canceled)

12. A sunscreen composition comprising:
a) from 5 to 50% by weight of at least one UV protective agent;
b) from 0.05 to 5% by weight of at least one hydrophobically modified clay mineral;
c) from 1 to 60% by weight of at least one oil component; and
d) from 0.1 to 4% by weight of at least one associative polymer.

13. The sunscreen composition according to claim 12, wherein the associative polymer is selected from the group consisting of silicone acrylate polymer; a copolymer of (meth)acrylic acid ester of ethoxylated C₈-C₃₀; a copolymer of itaconic acid ester of ethoxylated C₈-C₃₀; and a copolymer based on acrylamidomethylpropanesulphonic acid and (meth)acrylic ester of ethoxylated C₈-C₃₀, or C₁₀-C₃₀, or C₁₂-C₂₂, or C₁₂-C₁₈ fatty alcohol.

14. The sunscreen composition according to claim 12, wherein the associative polymer is selected from the group consisting of a copolymer of (meth)acrylic acid ester of ethoxylated C₈-C₃₀; a copolymer of itaconic acid ester of ethoxylated C₈-C₃₀; wherein the ethoxylated fatty alcohol in each case comprise 5 to 35 ethylene oxide radicals.

15. The sunscreen composition according to claim 12, wherein the associative polymer is selected from the group consisting of acrylates/beheneth-25 methacrylate copolymer, acrylates/steareth-20 methacrylate copolymer, acrylates/steareth-20 methacrylate crosspolymer, acrylated/steareth-20 itaconate copolymer; acrylate/ceteth-20 itaconate copolymer and acrylates/palmeth-25 itaconate copolymer, ammonium acryloyldimethyltaurate/beheneth-25-methacrylate copolymer, ammonium acryloyldimethyltaurate/beheneth-25 methacrylate crosspolymer, ammonium acryloyldimethyltaurate/Steareth-25 methacrylate crosspolymer, ammonium acryloyldimethyltaurate/steareth-8 methacrylate copolymer, ammonium acryloyldimethyltaurate/laureth-7 methacrylate copolyme, and a combination thereof.

16. The sunscreen composition according to claim 12, wherein the UV protective agent is selected from the group consisting of ethylhexyl methoxycinnamate, ethylhexyl triazone, methylene bis-benzotriazolyl tetramethylbutylphenol, bis-ethylhexyloxyphenol methoxyphenyl triazine, diethylamino hydroxybenzoyl hexyl benzoate, ethylhexyl salicylate and a combination thereof.

17. The sunscreen composition according to claim 12, wherein the hydrophobically modifed clay mineral is selected from the group consisting of stearalkonium bentonite, dimethyldistearammonium hectorite, disteardimonium hectorite, dimethylalkylammonium hectorite, benzyldimethylstearylammonium hectorite, magnesium aluminum silicate treated with distearyldimethylammonium chloride, and a combination thereof.

18. The sunscreen composition according to claim 12, wherein the oil component is selected from the group consisting of dicapryl carbonate, cetyl carbonate, cetearyl carbonate, stearyl carbonate, and a mixture thereof.

19. The sunscreen composition according to claim 12, further comprising one emulsifier.

20. The sunscreen composition according to claim 12, wherein the sunscreen composition is an emulsifier-free sunscreen composition.

21. A method of achieving an improved sunscreen protection effect, comprising applying the composition according to claim 12 onto the skin.

22. A method for protecting the skin comprising the composition as defined in claim 12.
